(19) United States
(12) Patent Application Publication
(10) Pub. No.: US 2026/0013752 A1
(43) Pub. Date: Jan. 15, 2026

(54) CATHETER DEVICE

(71) Applicant: TERUMO KABUSHIKI KAISHA, Tokyo (JP)

(72) Inventors: Satoshi SAWADA, Kanagawa (JP); Kobby GREENBERG, Misgav (IL); Arnon HADAS, Misgav (IL)

(73) Assignee: TERUMO KABUSHIKI KAISHA, Tokyo (JP)

(21) Appl. No.: 19/338,739

(22) Filed: Sep. 24, 2025

Related U.S. Application Data
(63) Continuation of application No. PCT/JP2024/008709, filed on Mar. 7, 2024.

(30) Foreign Application Priority Data
Mar. 24, 2023 (JP) 2023-047596

(51) Int. Cl. A61B 5/145; A61B 5/00; A61B 5/20; A61M 25/00

(52) U.S. Cl. CPC A61B 5/14507; A61B 5/14546; A61B 5/201; A61B 5/6852; A61M 25/0017; A61M 25/0023

(57) ABSTRACT

To stably measure a component in a biological fluid, an elongated catheter device is configured to bring the biological fluid flowing into a catheter into constant contact with a sensor surface. The elongated catheter device includes a lead-out lumen through which a biological fluid can flow; a lead-out port that allows an inside and an outside of the lead-out lumen to communicate; one or more sensor units capable of measuring a component contained in the biological fluid in the lead-out lumen; and a guide portion 40 that guides the biological fluid flowing into the lead-out lumen from the lead-out port toward the sensor unit, in which the guide portion protrudes in a radial direction orthogonal to a central axis of the catheter device in a longitudinal direction, and is disposed on a distal end side relative to the sensor unit.

### CROSS-REFERENCES TO RELATED APPLICATIONS

This application is a continuation of International Patent Application No. PCT/JP2024/008709 on Mar. 7, 2024, which claims priority to Japanese Patent Application No. 2023-047596 filed on Mar. 24, 2023, the entire content of both of which is incorporated herein by reference.

### TECHNOLOGICAL FIELD

The present invention generally relates to a catheter device that is indwelled in a living body and measures a target component from a biological fluid.

### BACKGROUND DISCUSSION

As a device for measuring a target component from a biological fluid in a state of being indwelled in a living body, for example, a catheter disclosed in JP 2021-62073 A is known.

The catheter of JP 2021-62073 A is a device that is indwelled in a bladder and measures an oxygen partial pressure in urine in the bladder, and includes a catheter main body having a lumen and a probe in which a sensor for measuring the oxygen partial pressure is disposed. In the catheter main body, a longitudinal hole through which the probe can penetrate in an axial direction of the catheter main body is formed at a distal end, and a pair of lateral holes for circulating urine stored in the bladder is formed. The catheter is used by inserting the catheter main body into the bladder and then slidingly moving the probe to be disposed at a predetermined position in the bladder.

### SUMMARY

In the device for measuring the target component from the biological fluid such as the catheter of Patent Literature 1, it is important to fix a sensor in the device to eliminate external factors as much as possible and to constantly measure the target component under the same conditions in order to perform highly accurate measurement. In addition, since the device continuously performs highly accurate measurement in real time, it is necessary to bring the biological fluid into constant contact with the sensor.

The catheter of Patent Literature 1 has a form in which the probe is slidingly moved to protrude into the bladder and used, but in order to eliminate the external factors as described above, it is also possible to perform measurement in a state where the probe is accommodated in the catheter main body.

However, in the catheter of Patent Literature 1, since an outer diameter of the catheter main body is small in order to pass through a urethra, and it is difficult to secure a lumen having a sufficient flow path width, there is a high possibility that urine flowing in from the longitudinal hole or the lateral hole stagnates in the flow path. In addition, in the catheter of Patent Literature 1, since the urine flowing in from the longitudinal hole or the lateral hole serving as a lead-out port linearly flows in a vertical direction, it is difficult to bring the urine into constant contact with a probe surface without stagnation.

At least one embodiment of the present invention has been made in view of the above circumstances, and specifically, it is an object of the present invention to provide a catheter device that enables stable measurement by bringing a biological fluid flowing into a catheter into constant contact with a sensor surface without stagnation around the sensor surface.

The object of the present invention is achieved by any one the following (1) to (17).

(1) An elongated catheter device configured to be indwelled in a living body, including: a lead-out lumen through which a biological fluid can flow; a lead-out port that allows an inside and an outside of the lead-out lumen to communicate; one or more sensor units capable of measuring a component contained in the biological fluid in the lead-out lumen; and a guide portion that guides the biological fluid flowing into the lead-out lumen from the lead-out port toward the sensor unit side, in which the guide portion protrudes in a radial direction orthogonal to a central axis of the catheter device in a longitudinal direction, and is disposed on a distal end side relative to the sensor unit.

(2) The catheter device according to (1), in which the guide portion is disposed on a proximal end side relative to a distal end side end portion of the lead-out port and on a distal end side relative to the sensor unit.

(3) The catheter device according to (1) or (2), in which the guide portion is disposed on a same side as the lead-out port with respect to the central axis in a cross-sectional view including the central axis of the catheter device.

(4) The catheter device according to (1) or (2), in which the guide portion is disposed on a side opposite to the lead-out port with respect to the central axis in a cross-sectional view including the central axis of the catheter device.

(5) The catheter device according to (1) or (2), in which the guide portion is disposed at a position not overlapping with a distal end of the sensor unit in an axial direction.

(6) The catheter device according to any one of (1) to (5), in which the guide portion has a length in which the guide portion extends until a radial length orthogonal to the central axis overlaps with the central axis in a cross-sectional view including the central axis of the catheter device.

(7) The catheter device according to any one of (1) to (6), in which the guide portion includes an upper surface facing a distal end side and a lower surface facing a proximal end side, and at least a central axis side end portion of the upper surface is inclined toward the proximal end side.

(8) The catheter device according to (7), in which the upper surface has a guide structure that guides the biological fluid flowing in from the lead-out port to the sensor unit side.

(9) The catheter device according to (1) or (2), in which the guide portion includes a wall portion covering the lead-out lumen and a hole portion formed in the wall portion and allowing the lead-out lumen on a distal end side of the guide portion and the lead-out lumen on a proximal end side of the guide portion to communicate, and the hole portion is disposed immediately above the sensor unit.

(10) The catheter device according to any one of (1) to (9) further including: a sensor lumen that liquid-tightly accommodates the sensor unit, in which the sensor unit includes a first sensor unit disposed so as to be partially exposed from the sensor lumen so as to be contactable with the biological fluid, and a second sensor unit liquid-tightly accommodated in the sensor lumen.

(11) The catheter device according to (10), in which the sensor lumen includes a first sensor lumen that accommodates the first sensor unit with at least a part thereof exposed, and a second sensor lumen that liquid-tightly accommodates the second sensor unit, and the guide portion is either a first blocking portion that blocks a distal end opening of the first sensor lumen or a second blocking portion that blocks a distal end opening of the second sensor lumen.

(12) The catheter device according to any one of (1) to (11), further including: a fixing portion that fixes the sensor unit, in which the fixing portion functions as the guide portion.

(13) The catheter device according to any one of (1) to (12), in which the guide portion includes a first guide portion, and a second guide portion disposed on a distal end side relative to the first guide portion and on a side opposite to the first guide portion with respect to the central axis in a cross-sectional view including the central axis of the catheter device.

(14) The catheter device according to any one of (1) to (13), in which the lead-out port includes a first lead-out port disposed on a same side as the guide portion with respect to the central axis and a second lead-out port disposed on a side opposite to the guide portion with respect to the central axis, in a cross-sectional view including the central axis of the catheter device.

(15) The catheter device according to any one of (1) to (14), in which the sensor unit includes a fluorescent sensor capable of measuring at least one kind of an oxygen partial pressure in the biological fluid, a carbon dioxide partial pressure in the biological fluid, an ion concentration in the biological fluid, a sodium ion in the biological fluid, a potassium ion in the biological fluid, glucose in the biological fluid, lactic acid in the biological fluid, and an antibody-added protein in the biological fluid.

(16) The catheter device according to (15), in which the living body is a bladder, and the biological fluid is urine.

(17) The catheter device according to any one of (1) to (16), further including: an indwelling portion that is indwelled in the living body and is capable of expanding and contracting, in which the guide portion is disposed on a distal end side relative to a proximal end side end portion of the indwelling portion.

(18) The catheter device according to any one of (1) to (17), further including: a sensor lumen that liquid-tightly accommodates the sensor unit, in which the sensor unit is insertable into and removable from the sensor lumen.

(19) The catheter device according to any one of (1) to (18), further including: a sensor lumen that liquid-tightly accommodates the sensor unit, in which the sensor lumen accommodates a transmission unit pulled out from the sensor unit, and the transmission unit includes at least one or more transmission unit connection points.

According to the catheter device of one embodiment, because the guide portion that comes into contact with the biological fluid flowing in from the lead-out port and guides the biological fluid to the sensor unit side is provided, the biological fluid flowing into the catheter constantly comes into contact with the sensor surface without stagnation and stable measurement can be performed.

According to another aspect, a catheter device configured to be indwelled in a living body comprises: an elongated tubular body having a distal end, the elongated tubular body including a lead-out lumen through which biological fluid from the living is flowable while the catheter device is indwelled in the living body; a lead-out port; a sensor configured to measure a component contained in biological fluid, and a guide portion. The lead-out lumen extends along the longitudinal extent of the elongated tubular body, and the lead-out port passes through the wall of the elongated tubular body and communicates the lead-out lumen with an outside of the elongated tubular body so that the biological fluid in the living body flows from the outside of the elongated tubular body into the lead-out lumen by way of the lead-out port while the catheter device is indwelled in the living body. The sensor is configured to measure a component contained in the biological fluid flowing in the lead-out lumen while the catheter device is indwelled in the living body, and the sensor is positioned in the lead-out lumen at a position on a proximal side of the distal end of the elongated tubular body. A transmission unit is connected to the sensor to convey information from the sensor to outside the catheter device. The guide portion: i) is positioned in the lead-out lumen; ii) is located on the proximal side of the lead-out port at a position to be contacted by the biological fluid flowing through the lead-out port and entering the lead-out lumen; iii) is located on a distal side of the sensor; iv) protrudes radially inwardly away from an inner surface of the elongated tubular body; and v) is configured to guide the biological fluid flowing through the lead-out port, entering the lead-out lumen and contacting the guide portion toward the sensor unit.

According to another aspect, a method comprises: inserting a catheter device into a biological lumen of a living body containing a biological fluid, wherein the catheter device comprises: a lead-out port; an axially extending lead-out lumen in fluid communication with the lead-out port; a sensor configured to measure a component contained in the biological fluid; and a guide portion. The method additionally includes the biological fluid in the biological lumen passing through the lead out port and is introduced into the lead out lumen after passing through the lead out port, and the biological fluid that is introduced into the lead out lumen by way of the lead out port contacts the guide portion that projects radially inwardly toward a central axis of the elongated catheter device and is guided by the guide portion toward the sensor.

### BRIEF DESCRIPTION OF THE DRAWINGS

FIG. 1 is a configuration view of a catheter device according to an embodiment that represents one example of the disclosed catheter.

FIG. 2 is a partial cross-sectional view of a periphery on a distal end side of the catheter device according to the present embodiment.

FIG. 3 is a cross-sectional view of a vicinity of a guide portion of the catheter device taken along the section line III-III in FIG. 2 in a direction orthogonal to a central axis as viewed in an axial direction.

FIG. 4A is a view illustrating a flow of a biological fluid (urine) flowing in from a lead-out port.

FIG. 4B is a view illustrating a flow of a biological fluid (urine) flowing in from the lead-out port.

FIG. 5A is a view illustrating an operation example of the catheter device according to the present embodiment.

FIG. 5B is a view illustrating an operation example of the catheter device according to the present embodiment.

FIG. 5C is a view illustrating an operation example of the catheter device according to the present embodiment.

FIG. 6 is a partial cross-sectional view of a periphery on a distal end side of a catheter device according to a first modification.

FIG. 7 is a partial cross-sectional view of a periphery on a distal end side of a catheter device according to a second modification.

FIG. 8 is a partial cross-sectional view of a periphery on a distal end side, which is an aspect of a catheter device according to a third modification.

FIG. 9 is a partial cross-sectional view of the periphery on the distal end side, which is another aspect of the catheter device according to the third modification.

FIG. 10 is a partial cross-sectional view of a periphery on a distal end side of a catheter device according to a fourth modification.

FIG. 11 is a partial cross-sectional view of a periphery on a distal end side, which is an aspect of a catheter device according to a fifth modification.

FIG. 12 is a partial cross-sectional view of the periphery on the distal end side of another aspect of the catheter device according to the fifth modification.

FIG. 13 is a partial cross-sectional view of a periphery on a distal end side, which is an aspect of a catheter device according to a sixth modification.

FIG. 14 is a partial cross-sectional view of the periphery on the distal end side, which is another aspect of the catheter device according to the sixth modification.

### DETAILED DESCRIPTION

Hereinafter, a mode for implementing the catheter device disclosed here will be described in detail with reference to the accompanying drawings. The embodiment herein is illustrated and described simply as an example of the disclosed catheter device and is not limiting in terms of the scope of the invention. Other forms, examples, technical operations, and the like that could be conceived by those skilled in the art without departing from the gist of the disclosure and are all included in the scope of the invention defined in the claims and the scope of equivalents thereof.

Furthermore, for the purpose of illustration and for ease of comprehension, the scale, aspect ratio, shape, and the like in the drawings attached to the present specification may be changed from actual ones as appropriate and illustrated schematically. The drawings are examples and do not limit the scope of the disclosure here.

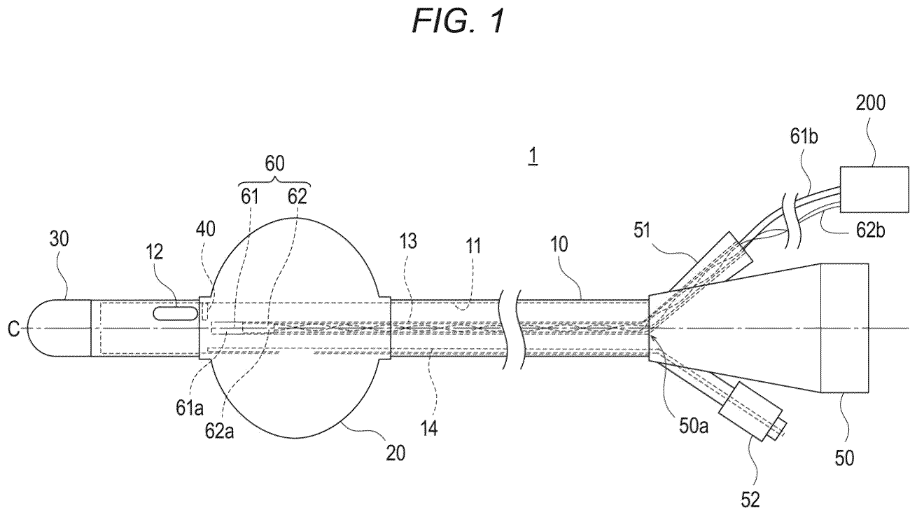

In the present specification, the following directions are defined for convenience of description. In FIG. 1, a “long axis direction (axial direction)” is a longitudinal direction (left-right direction in the drawing) of a catheter device 1 and is a direction along a central axis C of the catheter device 1. A “radial direction” is a direction away from or approaching the central axis C of the catheter device 1. A “peripheral direction” is a rotation direction with the central axis C of the catheter device 1 as a reference axis.

In FIG. 1, a “distal end” is a side to be inserted into a living body (left side in the drawing), and a side opposite to the distal end (side on which a hub 50 on the right side in the drawing is disposed) is a “proximal end”.

In the following description, ordinal numerals such as “first” and “second” are used, but are used for convenience's sake and do not define any order unless otherwise specified.

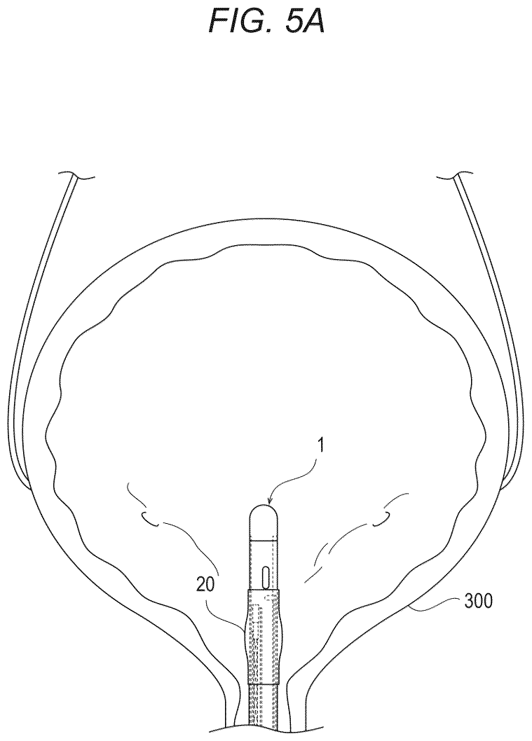

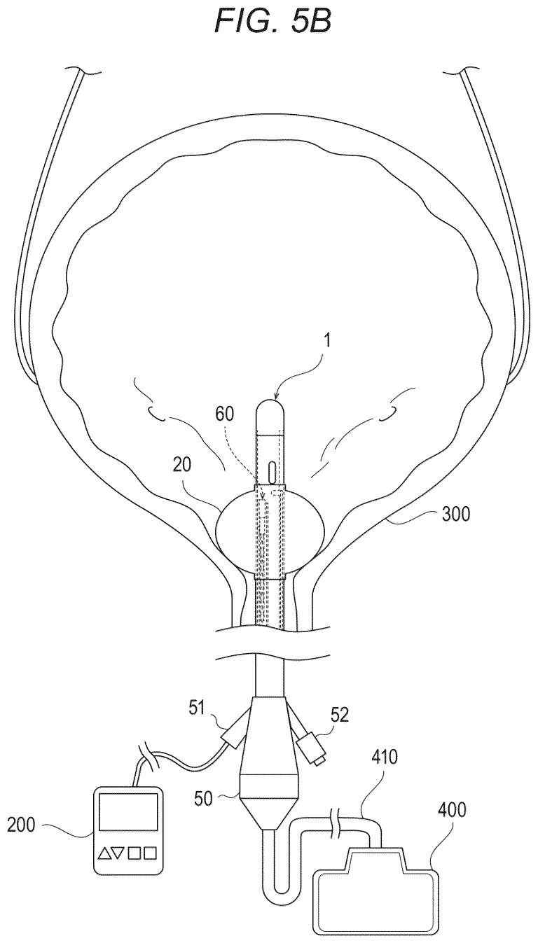

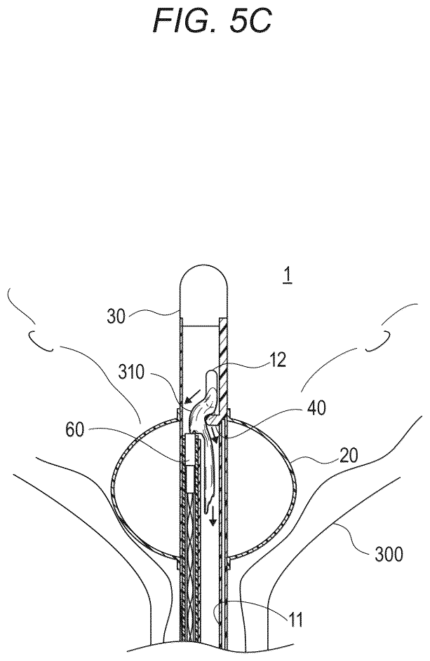

The catheter device 1 is a medical device that is partially indwelled in a living body, discharges a predetermined biological fluid from an inside of the living body to an outside of the living body, and measures biological information including a component to be measured in the biological fluid to monitor vitals of an organ. The catheter device 1 can be used, for example, as a device for indwelling an indwelling portion 20 as an indwelling site in a bladder, sequentially discharging urine 310 accumulated in a bladder 300 as the biological fluid from a lead-out port 12 formed in a main body portion 10, and measuring a partial pressure of oxygen in the urine 310 as a target component to be measured (see FIGS. 5A to 5C and the like).

The indwelling site of the catheter device 1 is not limited to the bladder 300, and may be in a biological lumen in which the catheter device 1 can be indwelled. In addition, the biological fluid to be measured by the catheter device 1 is not limited to the urine 310, and may be any fluid that can be discharged the outside of the living body and can contain a target component that can be biological information, or can exist in the living body where desired biological information can be measured.

### [Configuration]

A configuration of the catheter device 1 according to the present embodiment will be described.

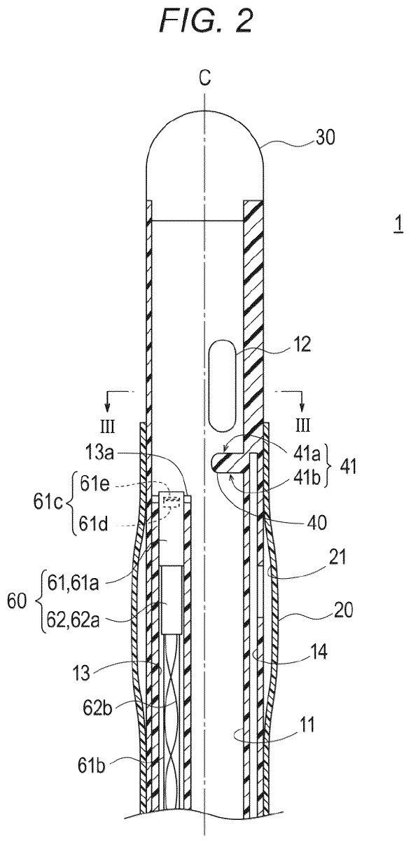

As illustrated in FIG. 1 or 2, the catheter device 1 includes an elongated main body portion 10, an indwelling portion 20 disposed on a distal end side of the main body portion 10, a distal end tip 30 disposed at a distal end of the main body portion 10, a guide portion 40 that guides a biological fluid (urine 310) flowing into a lead-out lumen 11 of the main body portion 10 through a lead-out port 12 in a predetermined direction, a hub 50 attached to a proximal end of the main body portion 10, and a sensor unit 60 that measures predetermined biological information including a target component in the biological fluid. As shown in FIGS. 1 and 2, the catheter device thus includes an elongated tubular body in which is disposed the guide portion. The elongated tubular body has a distal end, and the wall of the elongated tubular body surrounds the lead-out lumen 11.

### <Main Body Portion>

As illustrated in FIG. 1, the main body portion 10 is formed of an elongated tubular member having the lead-out lumen 11 communicating from the distal end side toward the proximal end side. The indwelling portion 20 is disposed on the distal end side of the main body portion 10. The distal end tip 30 is disposed at the distal end of the main body portion 10, and the proximal end thereof communicates with a distal end opening portion 50a of the hub 50.

The lead-out port 12 that allows the lead-out lumen 11 and an indwellable biological lumen (bladder 300) in which a biological fluid exists to communicate is formed on the distal end side of the main body portion 10 relative to the indwelling portion 20. In the present embodiment, the lead-out port 12 is formed on a side surface of the main body portion 10 closer to the indwelling portion 20. The formation position of the lead-out port 12 is not particularly limited as long as the lead-out lumen 11 and the bladder 300 can communicate with each other.

The main body portion 10 includes a sensor lumen 13 disposed in parallel with the lead-out lumen 11 in the axial direction. As illustrated in FIG. 2, the sensor lumen 13 has a distal end formed on the proximal end side relative to the lead-out port 12 in a longitudinal cross-sectional view including the central axis C.

The sensor lumen 13 accommodates a sensor unit 60 (an oxygen sensor main body 61a of an oxygen sensor 61 that is a first sensor unit, a temperature sensor main body 62a of a temperature sensor 62 that is a second sensor unit, and the like) including various sensors capable of measuring a target component to be measured in a biological fluid, and a transmission unit (an oxygen transmission unit 61b, a temperature transmission unit 62b, and the like) extending away from the sensor unit 60. The proximal end of the sensor lumen 13 communicates with a first branch portion 51 of the hub 50 with the proximal end side relative to the lead-out port 12 as a starting point. The sensor unit 60 may be disposed on the distal end side of the sensor lumen 13 or may be fixed to the distal end side.

As illustrated in FIG. 2, a blocking portion 13a extending in a direction intersecting the axial direction is formed at the distal end of the sensor lumen 13. The blocking portion 13a blocks a distal end opening of the sensor lumen 13. The oxygen sensor main body 61a or the oxygen transmission unit 61b in a state in which a part of the distal end side is exposed to the lead-out lumen 11 is fixed to the blocking portion 13a. On the other hand, the temperature sensor main body 62a is disposed or fixed to the sensor lumen 13 in a state of not being exposed to the lead-out lumen 11. The sensor lumen 13 can liquid-tightly accommodate the sensor unit 60 separately from the lead-out lumen 11 by the blocking portion 13a. In addition, the blocking portion 13a hinders the urine 310 from flowing into the sensor lumen 13, and prevents a failure, an electric shock, or the like of the sensor unit 60.

The main body portion 10 has a fluid lumen 14 through which a fluid for expanding and contracting the indwelling portion 20 flows. As illustrated in FIG. 2, a part of the fluid lumen 14 communicates with an expansion/contraction lumen 21 of the indwelling portion 20. The formation position of the fluid lumen 14 is not particularly limited, but in consideration of the influence on the sensor unit 60 due to the flow of the fluid, it is preferable to form the fluid lumen at a position away from the sensor unit 60, such as a thick portion on the side facing the sensor unit 60 in the radial direction.

Examples of a constituent material from which the main body portion 10 may be fabricated include, for example, polyolefins such as polyethylene, polypropylene, an ethylene-propylene copolymer, and an ethylene-vinyl acetate copolymer, thermoplastic resins such as soft polyvinyl chloride, various rubbers such as silicone rubber and latex rubber, various elastomers such as polyurethane elastomer, polyamide elastomer, and polyester elastomer, and crystalline plastics such as polyamide, nylon, polytetrafluoroethylene (PTFE), polyethylene terephthalate (PET), crystalline polyethylene, and crystalline polypropylene.

In these materials, for example, antithrombotic substances such as heparin, prostaglandin, urokinase, and arginine derivatives can be blended to obtain a material having antithrombogenicity. Furthermore, the main body portion 10 may be coated with a lubricious coating.

### <Indwelling Portion>

The indwelling portion 20 is disposed on the distal end side of the main body portion 10, and is indwelled at a predetermined indwelling position (bladder 300) in the living body. The indwelling portion 20 is a portion for maintaining an inserted state of the catheter device 1 in the living body. The indwelling portion 20 is formed of a balloon or the like that can expand and contract in response to the inflow and discharge of the fluid. The indwelling portion 20 prevents unintended removal of the catheter device 1 in a state of being indwelled in the bladder 300.

The indwelling portion 20 has the expansion/contraction lumen 21 through which a fluid flows in and out. The expansion/contraction lumen 21 is a lumen formed in the balloon. As illustrated in FIG. 2, a part of the expansion/contraction lumen 21 communicates with the fluid lumen 14, and the fluid flows in and out.

The constituent material from which the indwelling portion 20 may be fabricated is not particularly limited as long as it is a biocompatible material that can expand and contract by a fluid, and for example, the same material as that used for the main body portion 10 or a balloon of a known balloon catheter can be applied.

The indwelling portion 20 is not limited to the balloon, and may be differently configured to be indwellable in the living body and to prevent unintended removal of the catheter device 1.

### <Distal End Tip>

The distal end tip 30 is disposed at the distal end of the main body portion 10. When the catheter device 1 is advanced into the living body, the distal end tip 30 can suitably prevent damage or the like of a living body organ due to contact between the distal end and the living body organ (lumen inner wall or the like). The distal end tip 30 may include the lead-out port 12, the guide portion 40, and the sensor unit 60. In addition, the distal end tip 30 may include the lead-out port 12 and the guide portion 40. With such a configuration, it is not necessary to provide the guide portion 40 by processing the main body portion 10 having a small lumen diameter, and the guide portion 40 can be shaped more easily.

As a constituent material from which the distal end tip 30 may be fabricated, the same material as that of the main body portion 10 can be applied. In addition, the distal end tip 30 can be made of a material softer than the main body portion 10 from the viewpoint of preventing damage to an inner wall of a blood vessel. The distal end tip 30 may be made of the same material as the main body portion 10.

### <Guide Portion>

The guide portion 40 guides the urine 310 flowing in from the lead-out port 12 toward the distal end side of the sensor unit 60.

As illustrated in FIG. 2, the guide portion 40 is formed of a plate material protruding in the radial direction orthogonal to the central axis C side from an inner peripheral surface of the lead-out lumen 11, and includes bottom surfaces 41 including an upper surface 41a facing the distal end side and a lower surface 41b facing the proximal end side. The upper surface 41a of the guide portion 40 and a proximal end side end portion of the lead-out port 12 may have a flat surface continuous in the radial direction. With such a configuration, the creation of the lead-out port 12 and the creation of the guide portion 40 can be performed at the same time, and the guide portion 40 can be more easily shaped.

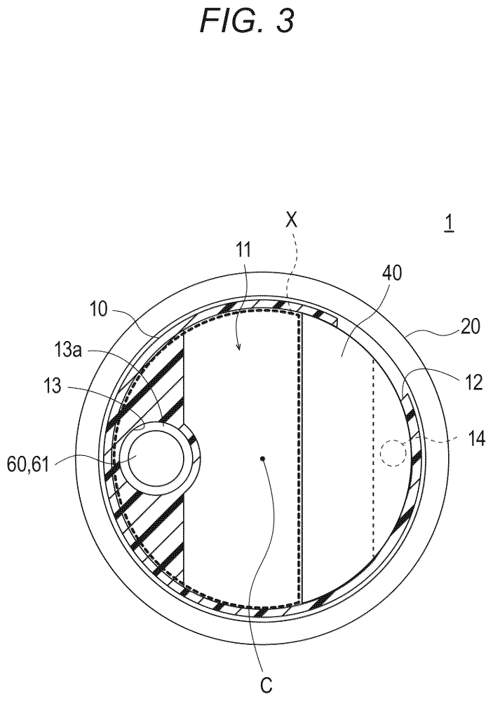

FIG. 3 is a transverse cross-sectional view of the catheter device 1 taken along the section line III-III in FIG. 2 in a direction orthogonal to the central axis C. A radial length of the guide portion 40 is a length that does not hinder discharge of the urine 310 flowing through the lead-out lumen 11 and can guide the urine 310 flowing in from the lead-out port 12 toward the distal end side of the sensor unit 60. In the transverse cross section of the main body portion 10 illustrated in FIG. 3 as viewed from the axial direction, a part of the lead-out lumen 11 is blocked by the guide portion 40 on the right side in the drawing, and in a region X surrounded by a thick dotted line on the left side in the drawing, the upper side of the sensor unit 60 is opened such that the guide portion 40 is not disposed and the lead-out lumen 11 communicates in the axial direction. Therefore, the flow direction of the urine 310 flowing in from the lead-out port 12 changes to the distal end side of the sensor unit 60 after coming into contact with the guide portion 40.

The guide portion 40 is disposed on the distal end side relative to the sensor unit 60 in order to guide the urine 310 flowing in from the lead-out port 12 to the sensor unit 60. The guide portion 40 is disposed on the distal end side relative to a proximal end side end portion of the indwelling portion 20. From the viewpoint of promoting efficient guidance of the urine 310, the guide portion 40 is preferably disposed on the proximal end side relative to the distal end side end portion of the lead-out port 12 and on the distal end side relative to the sensor unit 60.

The guide portion 40 can be disposed on the same side as the lead-out port 12 or on the side opposite to the lead-out port 12 with respect to the central axis C in a cross-sectional view including the central axis C of the main body portion 10. That is, the guide portion 40 may be positioned on the same side as the lead-out port 12 or on the side opposite to the lead-out port 12 with respect to the central axis C as seen in a cross-sectional view taken along a plane that contains the central axis of the catheter device.

Further, the guide portion 40 may be disposed at a position not overlapping with the distal end of the sensor unit 60 in the axial direction. With such a configuration, the guide portion 40 can efficiently guide the urine 310 flowing in from the lead-out port 12 to the distal end side of the sensor unit 60.

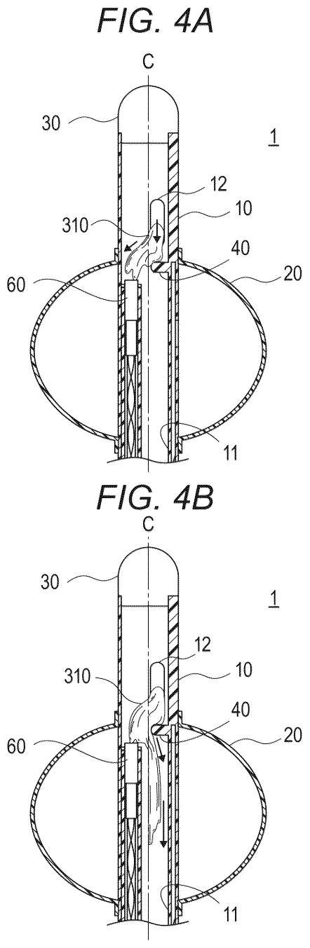

FIGS. 4A and 4B illustrate a flow of the urine 310 flowing into the catheter device 1 through the lead-out port 12. As illustrated in FIG. 4A, the urine 310 flowing in from the lead-out port 12 comes into contact with the guide portion 40 so that a flow direction of the urine 310 is converted from a vertical direction (axial direction) to a direction intersecting the vertical direction. As illustrated in FIG. 4B, the flow direction of the urine 310 that has come into contact with the guide portion 40 is directed to the sensor unit 60 by the guide portion 40, and after coming into contact with the distal end of the sensor unit 60, the urine flows to the proximal end side. Since the guide portion 40 can change the flow direction of the urine 310 to a direction intersecting the vertical direction, the urine 310 can be guided to the distal end of the sensor unit 60 without stagnation in the lead-out lumen 11.

In a conventional urinary catheter, urine flowing in from a lead-out port formed in a main body portion of the catheter linearly flows in a vertical direction from the lead-out port, and thus, is less likely to come into contact with a sensor unit disposed on an inner peripheral surface side of the main body portion. In order to bring the sensor unit and urine into contact with each other, it is necessary to design the catheter in consideration of the arrangement of the sensor unit and the lead-out port, but since the catheter is a device having a small lumen diameter, it is difficult to manufacture the catheter due to many restrictions. Even when the lead-out port is disposed at a position where the lead-out port can be brought into contact with the sensor unit, since urine flows linearly in the vertical direction from the lead-out port, there arises a new problem that stagnation is likely to occur at a corner portion or the like of the sensor unit with which urine is brought into contact. On the other hand, since the catheter device 1 of the present embodiment includes the guide portion 40, as illustrated in FIGS. 4A and 4B, after the urine 310 flowing in from the lead-out port 12 comes into contact with the guide portion 40 and is guided to the sensor unit 60 side without stagnation, and thus the urine 310 immediately after flowing in constantly comes into contact with the sensor surface. Therefore, the catheter device 1 can stably measure the urine 310 immediately after flowing in from the lead-out port 12.

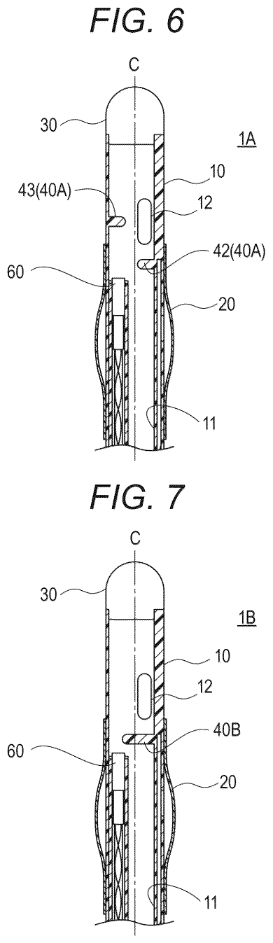

The shape, the number of formation, and the formation position of the guide portion 40 are not particularly limited as long as it is configured to be able to guide the urine 310 flowing in from the lead-out port 12 to the distal end side of the sensor unit 60 by protruding from at least the inner peripheral surface of the lead-out lumen 11 in the radial direction orthogonal to the central axis C side. As a preferable form of the guide portion 40, in addition to the form of being disposed in the vicinity of the proximal end side end portion of the lead-out port 12 as illustrated in FIG. 2, forms of respective modifications (first modification to fifth modification and the like) as illustrated in FIGS. 6 to 12 can be exemplified.

### <Hub>

The hub 50 is configured to be connectable to a connector (catheter connector, uro-connector, Y-connector, Luer connector, and the like) known in the medical field, and can be discharged to a urine collection bag 400 through a urine collection tube 410 connected via the connector (see FIG. 5B). The distal end opening portion of the hub 50 is connected to the proximal end portion of the main body portion 10, and the lead-out lumen 11 communicates with the distal end opening portion of the hub 50. As a result, the urine 310 flowing in from the lead-out port 12 flows through the lead-out lumen 11 and is discharged to the outside of the living body from the hub 50.

As illustrated in FIG. 1, the hub 50 includes a first branch portion 51 for housing and directing the transmission unit of the sensor unit 60 to the outside of the living body. In the first branch portion 51, the transmission unit of the sensor unit 60 may be inserted or positioned slidably in the axial direction.

In addition, as illustrated in FIG. 1, the hub 50 has a second branch portion 52 in communication with the fluid lumen 14 for allowing the inflow and discharge of the fluid to and from the expansion/contraction lumen 21 of the indwelling portion 20 to the outside of the living body. The second branch portion 52 is configured to be connected to an indeflator (inflation device) or a syringe to connect the indwelling portion 20 to a device capable of expanding and contracting the indwelling portion 20. A one-way valve may be provided in the second branch portion 52 so that the indwelling portion 20 can maintain the state of expansion and contraction. By doing so, the expanded state can be maintained even when the syringe is detached.

In the catheter device 1 of the present embodiment, the hub 50 includes the first branch portion 51 and the second branch portion 52 as illustrated in FIG. 1. However, the arrangement positions of the first branch portion 51 and the second branch portion 52 are not limited to the hub 50, and at least one of the first branch portion 51 and the second branch portion 52 may be formed on a proximal end side outer peripheral surface of the main body portion 10 so as to communicate with the lumen to be connected. When both the first branch portion 51 and the second branch portion 52 are formed on the proximal end side outer peripheral surface of the main body portion 10, the hub 50 functions as a member that communicates only with the lead-out lumen 11 to discharge the urine 310 to the outside of the living body.

### <Sensor Unit>

The sensor unit 60 acquires predetermined biological information including a target component in a biological fluid. The sensor unit 60 can include at least one or more sensors such as the oxygen sensor 61 and the temperature sensor 62. Furthermore, the sensor unit 60 is not limited to the above-described sensors, and can include, for example, a sensor capable of acquiring a target component in a biological fluid or predetermined biological information, such as a flow velocity sensor, an optical sensor, an ultrasonic sensor, a pressure sensor, a flow rate sensor, an acceleration sensor, a conductivity sensor, or a biomarker sensor. The sensor unit 60 (the oxygen sensor 61 and the temperature sensor 62) may be accommodated in a state of being fixed to the sensor lumen 13 in advance, or may be accommodated so as to be insertable into and removable from the sensor lumen 13. When the sensor unit 60 is configured to be accommodated so as to be insertable into and removable from the sensor lumen 13, replacement of the sensor unit 60 is facilitated, and maintainability is improved. In addition, since the sensor unit 60 can be inserted after catheter insertion or the sensor unit 60 can be removed before catheter removal, operability is improved.

The oxygen sensor 61 is configured as a so-called known fluorescent (optical) oxygen sensor, and is configured to emit fluorescence having an intensity corresponding to an oxygen concentration contained in the urine 310 by receiving irradiation of excitation light. The oxygen sensor 61 includes the oxygen sensor main body 61a that reacts with oxygen contained in the urine 310 and the oxygen transmission unit 61b optically connectable to the oxygen sensor main body 61a.

A measurement unit 61c capable of optically measuring oxygen in the urine 310 is disposed at a distal end of the oxygen sensor main body 61a. As illustrated in FIG. 2, the measurement unit 61c includes a substrate 61d having optical transparency, and a phosphor 61e applied to substantially the entirety of one surface of substrate 61d. Furthermore, in order to prevent deterioration of a fluorescent substance due to ambient light, a light shielding layer that is gas-permeable and capable of shielding light may be formed on an upper surface of the phosphor 61e.

As illustrated in FIG. 2, the oxygen sensor main body 61a or the oxygen transmission unit 61b is disposed with a part thereof fixed to the blocking portion 13a so that the measurement unit 61c at the distal end is exposed in the lead-out lumen 11.

The oxygen transmission unit 61b is constituted by an optical fiber, and optically connected to the measurement unit 61c of the oxygen sensor main body 61a and a measurement device 200 (see FIG. 5B). The oxygen transmission unit 61b is capable of irradiating the phosphor 61e with excitation light and receiving fluorescence from the phosphor 61e, and is fixed to the sensor lumen 13 in a state where the distal end surface thereof is positioned with respect to the phosphor 61e. The oxygen transmission unit 61b irradiates the phosphor 61e with excitation light from a light source unit of the measurement device 200, and transmits fluorescence emitted from the phosphor 61e excited by the excitation light to a light receiving unit of the measurement device 200. The oxygen transmission unit 61b, which is a transmission unit, may be constituted by one optical fiber, or a single transmission unit may be constituted by connecting a plurality of optical fibers. That is, the transmission unit (the oxygen transmission unit 61b) may be connected through a transmission unit connection point formed at a connection portion of the plurality of transmission units 61b. The transmission unit connection point is a portion where the two transmission units 61b are connected to each other, and the connection between the transmission units 61b can be detachably connected by connector connection or the like. Since the transmission unit is provided with the transmission unit connection point, the transmission unit can be easily replaced, and the replaced transmission unit can be reused, so that a cost reduction effect can be obtained and maintainability can be improved. In addition, it is possible to improve operability at the time of insertion and removal of the catheter while preventing damage to the transmission unit at the time of insertion and removal of the catheter.

The temperature sensor 62 is capable of measuring a temperature in the bladder 300, and more specifically measures a temperature of the urine 310 within the bladder 300. The temperature sensor 62 may measure the temperature of the urine 310 flowing through the lead-out lumen 11. The temperature sensor 62 includes the temperature sensor main body 62a that measures the temperature of the urine 310 and the temperature transmission unit 62b that can be electrically connected to the temperature sensor main body 62a.

The temperature sensor main body 62a is constituted by a device capable of measuring a temperature such as a thermocouple, a resistance temperature detector, or a thermistor, and is accommodated in the sensor lumen 13 in a state of being fixed to or being in close contact with any part of the distal end outer peripheral surface of the oxygen transmission unit 61b by the blocking portion 13a. Unlike the oxygen sensor main body 61a, the temperature sensor main body 62a is desirably disposed such that the distal end or the distal end surface thereof is not exposed to the lead-out lumen 11, that is, not in direct contact with the urine 310. Alternatively, the temperature sensor main body 62a may be disposed on the proximal end side relative to the oxygen sensor main body 61a so that the distal end or the distal end surface thereof is not exposed to the lead-out lumen 11. More specifically, the temperature sensor main body 62a may be disposed on the proximal end side relative to the oxygen sensor main body 61a and adjacent to the oxygen sensor main body 61a in a direction orthogonal to the central axis C of the catheter device 1, and may be disposed such that the distal end or the distal end surface thereof is not exposed to the lead-out lumen 11. As a result, the temperature sensor 62 can be liquid-tightly accommodated in the sensor lumen 13 separately from the lead-out lumen 11, so that the inflow of the urine 310 into the sensor lumen 13 is hindered in a way that prevents a failure, an electric shock, or the like of the sensor unit 60. The temperature sensor main body 62a can measure the temperature in the bladder 300, and measures the temperature of the urine 310 in the bladder 300.

The temperature sensor main body 62a may measure the temperature of the urine 310 flowing through the lead-out lumen 11. Two or more temperature sensor main bodies 62a may be provided in the sensor lumen 13, and by providing two or more temperature sensor main bodies, for example, a heater is provided between the two temperature sensor main bodies 62a, and the temperature sensor 62 can be used as a thermal flowmeter.

The temperature transmission unit 62b is electrically connected to the temperature sensor main body 62a, and transmits an electric signal based on a temperature value of the urine 310 measured by the temperature sensor main body 62a to the measurement device 200. The temperature transmission unit 62b is fixed to any part of the outer peripheral surface of the oxygen transmission unit 61b or fixed in a state of being in close contact with the blocking portion 13a, and the proximal end thereof is connected to the measurement device 200 through the first branch portion 51 (see FIG. 5B).

### [Operation]

Next, an operation at the time of indwelling the catheter device 1 will be described with reference to FIGS. 5A to 5C as appropriate.

As illustrated in FIG. 5A, an operator inserts the catheter device 1 from an external urethral opening and delivers the indwelling portion 20 into the bladder 300.

Subsequently, as illustrated in FIG. 5B, the operator causes a fluid to flow from the second branch portion 52 to expand the indwelling portion 20. When the expansion of the indwelling portion 20 is confirmed, the operator connects the transmission unit (the oxygen transmission unit 61b and the temperature transmission unit 62b) of the sensor unit 60 to the measurement device 200 through the first branch portion 51, and connects the urine collection tube 410 to the hub 50. As a result, indwelling processing of the catheter device 1 in the living body is completed. The connection between the transmission unit of the sensor unit 60 and the measurement device 200 and the connection between the hub 50 and the urine collection tube 410 may be performed in advance before the catheter device 1 is inserted into the bladder.

After indwelling the catheter device 1, the urine 310 in the bladder 300 flows in from the lead-out port 12, comes into contact with the guide portion 40 and is guided to the distal end side of the sensor unit 60, comes into contact with the distal end portion of the sensor unit 60, and then is guided to the outside of the living body through the lead-out lumen 11 as illustrated in FIG. 5C. The catheter device 1 measures a partial pressure of oxygen in the urine 310 that has come into contact with the oxygen sensor 61 of the sensor unit 60, and measures the temperature of the urine 310 in the bladder 300 with the temperature sensor 62 to acquire biological information. The partial pressure of oxygen is corrected by the temperature acquired by the temperature sensor 62, and more accurate measurement becomes possible.

### Modified Examples

The catheter device 1 can be appropriately converted and implemented as in the following modifications. In the following description of each of the modifications, differences from the above-described form will be mainly described, and constituent elements having functions equivalent to those of other forms or constructions will be denoted by the same or related reference numerals and will not be described again in detail. In addition, the configuration, the members, the method of use, and the like may be the same as those in each form. Furthermore, each modification can be implemented by appropriately selecting a necessary configuration among the configurations indicated in each modification and combining the configuration with other forms within a range not departing from the gist of the disclosure here.

### <First Modification>

A catheter device 1A of a first modification includes guide portions 40A. The guide portions 40A are the same in shape and the like as the guide portion 40 of the catheter device 1 described above, but a difference resides in that the number of guide portions formed in the lead-out lumen 11 is plural.

As illustrated in FIG. 6, the guide portions 40A include a first guide portion 42 disposed on the proximal end side relative to the proximal end side end portion of the lead-out port 12, and a second guide portion 43 disposed on the distal end side relative to the first guide portion 42 and on the side opposite to the first guide portion 42 with respect to the central axis C in a cross-sectional view including the central axis C of the main body portion 10.

The second guide portion 43 is preferably disposed at the position illustrated in FIG. 6 in consideration of the flow of the biological fluid flowing in from the lead-out port 12. That is, the second guide portion 43 is preferably disposed on the distal end side relative to the first guide portion 42 and on the proximal end side relative to the distal end side end portion of the lead-out port 12. However, the arrangement position of the second guide portion 43 is not particularly limited as long as it guides the biological fluid flowing in from the lead-out port 12 to the sensor unit 60 and does not hinder the flow of the biological fluid to the outside of the living body.

The guide portion 40A is not particularly limited as long as it guides at least the biological fluid flowing in from the lead-out port 12 to the sensor unit 60, and it is formed in such a number as not to hinder the flow of the biological fluid to the outside of the living body. As illustrated in FIG. 6, the number of guide portions 40A may be two or three or more.

Since the catheter device 1A of the first modification includes the guide portions 40A having the above-described configuration, the urine 310 is guided toward the sensor unit 60 without stagnation from the lead-out port 12 to the sensor unit 60, and the urine 310 can be constantly brought into contact with the sensor unit 60. In addition, since the catheter device 1A includes the first guide portion 42 and the second guide portion 43, the flow direction of the urine 310 can be effectively converted to the distal end side of the sensor unit 60 to bring the urine 310 into contact with the sensor unit 60.

### <Second Modification>

As illustrated in FIG. 7, a catheter device 1B of a second modification includes a guide portion 40B. The guide portion 40B is different in shape from other forms.

The guide portion 40B has a length in which the guide portion extends until a radial length orthogonal to the central axis C overlaps with (intersects or extends beyond) the central axis C in a cross-sectional view including the central axis C of the main body portion 10. Since the guide portion 40B is disposed on the proximal end side relative to the distal end side end portion of the lead-out port 12, it is possible to guide the urine 310 flowing in from the lead-out port 12 to the vicinity of the upper portion of the sensor unit 60.

The length of the guide portion 40B is not particularly limited as long as the flow of urine 310 can be guided at least toward the distal end of the sensor unit 60.

Since the catheter device 1B of the second modification includes the guide portion 40B having the above-described configuration, the urine 310 is guided toward the sensor unit 60 without stagnation from the lead-out port 12 to the sensor unit 60, and the urine 310 can be constantly brought into contact with the sensor unit 60. In addition, the catheter device 1B has a length in which the guide portion 40B extends until the radial length orthogonal to the central axis C overlaps with the central axis C in the cross-sectional view including the central axis C of the main body portion 10, and thus, it is possible to effectively guide the urine 310 flowing in from the lead-out port 12 to the distal end side of the sensor unit 60.

### <Third Modification>

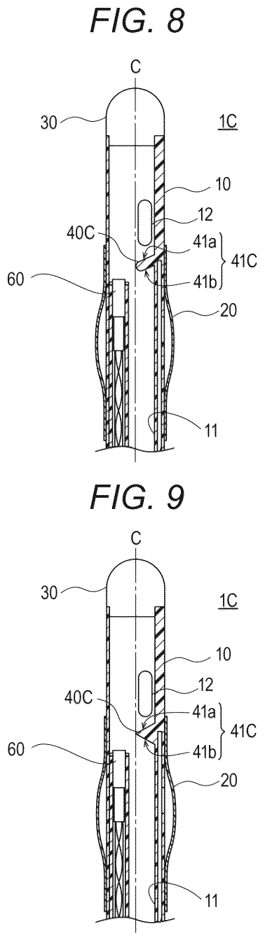

A catheter device 1C of a third modification includes a guide portion 40C as illustrated in FIGS. 8 and 9. The guide portion 40C is different in shape from other forms.

In the guide portion 40C, a central axis side end portion of the upper surface 41a on the lead-out port 12 side is inclined toward the proximal end side. That is, the upper surface 41a of the guide portion 40C has an inclined surface inclined at a predetermined angle toward the distal end side of the sensor unit 60. The guide portion 40C is disposed on the proximal end side relative to the distal end side end portion of the lead-out port 12. Therefore, when the urine 310 flowing in from the lead-out port 12 comes into contact with the upper surface 41a, the urine flows directly along the inclined upper surface 41a to the distal end side of the sensor unit 60 without stagnation.

As illustrated in FIG. 8, the guide portion 40C can be formed by a plate-like member extending obliquely toward the proximal end side from the vicinity of the proximal end side end portion of the lead-out port 12 toward the distal end side of the sensor unit 60. In the form illustrated in FIG. 8, in the guide portion 40C, the central axis side end portions of the upper surface 41a and the lower surface 41b which are the bottom surfaces 41C on the distal end side are inclined toward the proximal end side.

The guide portion 40C may be configured such that at least the central axis side end portion of the upper surface 41a is inclined toward the proximal end side. Therefore, as illustrated in FIG. 9, it is also possible to form a triangular cross section inclined from the vicinity of the proximal end side end portion of the lead-out port 12 toward the distal end side of the sensor unit 60.

The inclination angle of the bottom surface 41C of the guide portion 40C is not particularly limited as long as at least the urine 310 flowing in from the lead-out port 12 can be guided to the distal end side of the sensor unit 60. Therefore, the inclination angle of the bottom surface 41C of the guide portion 40C can be appropriately set according to the positional relationship between the lead-out port 12 and the sensor unit 60. The inclination angle of the bottom surface 41C is preferably 5° to 80°, and more preferably 15° to 60° with respect to the central axis C so that the central axis side end portion of the upper surface 41a is inclined toward the proximal end side.

However, the inclination angle of the lower surface 41b of the guide portion 40C is not limited to the above angular range.

The bottom surface 41C may be a smooth surface as a whole as illustrated in FIGS. 8 and 9, or may have a guide structure in which the urine 310 flowing in from the lead-out port 12 is efficiently guided to the sensor unit 60 side. The guide structure is a structure in which the flow direction of the urine 310 that has come into contact with the upper surface 41a is intentionally changed to the sensor unit 60 side, and specific examples thereof include a configuration in which one or more protrusions are provided on the upper surface 41a, a configuration in which the upper surface 41a has a multi-step stair shape, and a configuration in which a groove whose width narrows toward the central axis side end portion of the upper surface 41a is provided.

Since the catheter device 1C of the third modification includes the guide portion 40C having the above-described configuration, the urine 310 is guided toward the sensor unit 60 without stagnation from the lead-out port 12 to the sensor unit 60, and the urine 310 can be constantly brought into contact with the sensor unit 60. In the catheter device 1C, since the guide portion 40C has the inclined surface inclined toward the proximal end side toward the distal end side of the sensor unit 60, the urine 310 flowing in from the lead-out port 12 can be effectively guided to the distal end side of the sensor unit 60.

### <Fourth Modification>

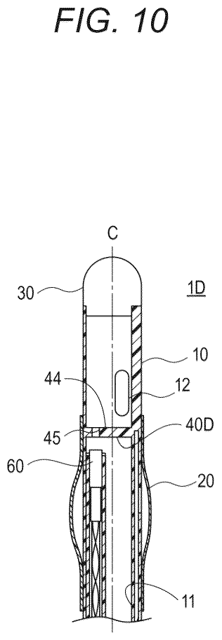

As illustrated in FIG. 10, a catheter device 1D of a fourth modification includes a guide portion 40D. The guide portion 40D is different in shape from other forms.

The guide portion 40D includes a wall portion (wall) 44 covering the lead-out lumen 11 and a hole portion (through hole) 45 formed in the wall portion 44 and allowing the lead-out lumen 11 on the distal end side of the guide portion 40D and the lead-out lumen 11 on the proximal end side of the guide portion 40D to communicate. The hole portion 45 is disposed above the sensor unit 60.

The wall portion 44 is disposed so as to block the lead-out lumen 11 on the distal end side relative to the distal end of the sensor unit 60. The wall portion 44 converts the flow direction of the urine 310 flowing in from the lead-out port 12 into a direction intersecting the inflow direction. For example, when the urine 310 flows in the vertical direction (lower direction and axial direction in FIG. 10) from the lead-out port 12, the wall portion 44 can temporarily receive the urine 310 and convert the flow direction of the urine 310 into a direction (left-right direction in FIG. 10 or the like) intersecting the vertical direction. As illustrated in FIG. 10, when the wall portion 44 is disposed on the proximal end side relative to the proximal end side end portion of the lead-out port 12 and on the distal end side relative to the distal end of the sensor unit 60, the urine 310 flowing in from the lead-out port 12 can be smoothly guided from the hole portion 45 to the distal end side of the sensor unit 60.

The hole portion 45 is formed to penetrate the wall portion 44 in the axial direction, and guides the urine 310 that has come into contact with the wall portion 44 to the distal end side of the sensor unit 60. The shape, the size, the number of formation, and the like of the hole portion 45 are not particularly limited as long as the urine 310 flowing along the wall portion 44 can be smoothly guided to the sensor unit 60.

The surface of the wall portion 44 on the lead-out port 12 side may have a structure for allowing the urine 310 flowing in from the lead-out port 12 to easily flow toward the hole portion 45. Examples of such a structure include a configuration in which at least a part of the upper surface facing the distal end side of the wall portion 44 that comes into contact with the urine 310 is inclined from the lead-out port 12 toward the hole portion 45, and a configuration in which one or more recessed grooves extending from below the lead-out port 12 toward the hole portion 45 are provided on the upper surface of the wall portion 44.

Since the catheter device 1D of the fourth modification includes the guide portion 40D having the above-described configuration, the urine 310 is guided toward the sensor unit 60 without stagnation from the lead-out port 12 to the sensor unit 60, and the urine 310 can be constantly brought into contact with the sensor unit 60. In addition, in the catheter device 1D, the hole portion 45 formed in the wall portion 44 is disposed immediately above the distal end portion of the sensor unit 60.

Therefore, the urine 310 flowing in from the lead-out port 12 can be efficiently guided to the distal end portion of the sensor unit 60.

### <Fifth Modification>

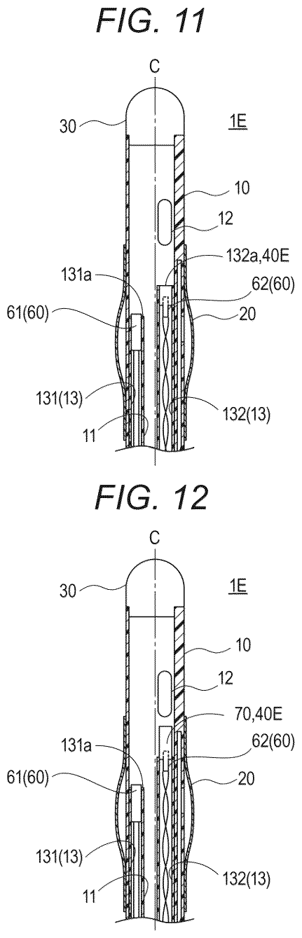

As illustrated in FIG. 11, a catheter device 1E of a fifth modification includes a guide portion 40E. The guide portion 40E is different from the other forms in that a member such as a protrusion is not disposed as in each of the above-described modifications, and a distal end surface of the sensor lumen accommodating the sensor unit 60 is used as the guide portion 40E.

The guide portion 40E is the blocking portion 13a (a first blocking portion 131a or a second blocking portion 132a) that blocks the distal end opening of the sensor lumen 13.

The sensor lumen 13 includes a first sensor lumen 131 that accommodates the oxygen sensor 61 with a part including the distal end thereof exposed, and a second sensor lumen 132 that liquid-tightly accommodates the temperature sensor 62.

The first sensor lumen 131 has the first blocking portion 131a at the distal end. The first blocking portion 131a holds the oxygen sensor 61 in a state where a part including the distal end of the oxygen sensor 61 is exposed from the distal end surface of the first blocking portion 131a, and blocks the distal end opening of the first sensor lumen 131.

As illustrated in FIG. 11, in the first sensor lumen 131, the first blocking portion 131a is disposed on the proximal end side relative to the second blocking portion 132a on the side opposite to the lead-out port 12 with respect to the central axis C in a cross-sectional view including the central axis C of the main body portion 10.

The second sensor lumen 132 has the second blocking portion 132a at the distal end. The second blocking portion 132a holds at least a part of the temperature sensor 62 in a state where the temperature sensor 62 is liquid-tightly accommodated, and blocks the distal end opening of the second sensor lumen 132. The temperature sensor 62 is liquid-tightly accommodated in the second sensor lumen 132 without being exposed from the distal end surface of the second blocking portion 132a.

As illustrated in FIG. 11, in a cross-sectional view including the central axis C of the main body portion 10, the second sensor lumen 132 is disposed on the same side as the lead-out port 12 with respect to the central axis C, with the second blocking portion 132a disposed on the distal end side relative to the first blocking portion 131a and below the lead-out port 12, that is, on the proximal end side relative to the proximal end side end portion of the lead-out port 12.

By disposing the first sensor lumen 131 and the second sensor lumen 132 in the arrangement illustrated in FIG. 11 (the first blocking portion 131a is disposed on the proximal end side relative to the second blocking portion 132a, and the second blocking portion 132a is disposed on the distal end side relative to the first blocking portion 131a and on the proximal end side relative to the proximal end side end portion of the lead-out port 12), the second blocking portion 132a of the second sensor lumen 132 functions as the guide portion 40E that comes into contact with the urine 310 flowing in from the lead-out port 12 and then guides the urine to the oxygen sensor 61 held by the first sensor lumen 131.

The guide portion 40E may cause the first blocking portion 131a of the first sensor lumen 131 to function as the guide portion 40E. In this case, the arrangement of the first sensor lumen 131 and the second sensor lumen 132 is opposite to that in FIG. 11 with respect to the central axis C.

In addition, the guide portion 40E can include a fixing portion 70 that fixes the sensor unit 60 to the catheter device 1E.

As illustrated in FIG. 12, at least a part of the fixing portion 70 can be disposed to protrude from the distal end opening of the sensor lumen 13. The fixing portion 70 is fixed to the inner peripheral surface of the lead-out lumen 11 in a state of holding at least a part of the sensor unit 60.

The fixing portion 70 illustrated in FIG. 12 holds and fixes the temperature sensor 62 to the catheter device 1 while blocking the second sensor lumen 132.

Similarly to the second blocking portion 132a illustrated in FIG. 11, when the urine 310 flowing in from the lead-out port 12 comes into contact with the fixing portion 70, the flow direction of the urine 310 is converted into the distal end side of the oxygen sensor 61, and the urine 310 can be brought into contact with the distal end side of the oxygen sensor 61.

As illustrated in FIG. 12, the fixing portion 70 may hold the sensor unit 60 with at least a part including the distal end portion of the sensor unit 60 exposed, or may hold the entire distal end portion of the sensor unit 60 in a buried state. In addition, a sensor (for example, the oxygen sensor 61 or the like) other than the temperature sensor 62 may be fixed to the fixing portion 70.

Since the catheter device 1E of the fifth modification includes the guide portion 40E having the above-described configuration, the urine 310 is guided toward the sensor unit 60 without stagnation from the lead-out port 12 to the sensor unit 60, and the urine 310 can be constantly brought into contact with the sensor unit 60. In addition, the catheter device 1E can guide the urine 310 to the sensor unit 60 to which the urine 310 is to be guided by using any one of the first blocking portion 131a of the first sensor lumen 131 and the second blocking portion 132a of the second sensor lumen 132 individually accommodating the sensor unit 60, or the fixing portion 70, so that the number of parts is reduced and the configuration can be simplified.

### <Sixth Modification>

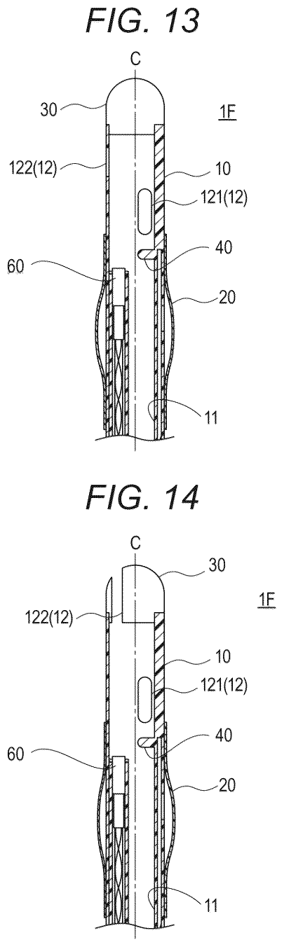

As illustrated in FIGS. 13 and 14, a catheter device 1F of a sixth modification is different from the other forms in including a plurality of lead-out ports 12. The guide portion 40 can appropriately adopt each mode described above.

As illustrated in FIG. 13, the catheter device 1F has the lead-out ports 12 including a first lead-out port 121 and a second lead-out port 122.

As illustrated in FIG. 13, the first lead-out port 121 and the second lead-out port 122 can be formed on the side surface of the main body portion 10, respectively. In addition, as illustrated in FIG. 14, the second lead-out port 122 can be formed to extend in the axial direction so that the outer surface of the distal end tip 30 disposed at the distal end of the main body portion 10 communicates with the lead-out lumen 11. As illustrated in FIGS. 13 and 14, the first lead-out port 121 and the second lead-out port 122 are preferably disposed on the opposite sides with respect to the central axis C in a cross-sectional view including the central axis C of the main body portion 10 in order to allow the urine 310 to efficiently flow in.

The shape, the number of formation, the arrangement position, and the like of the lead-out ports 12 are not particularly limited as long as the urine 310 can efficiently flow into the lead-out lumen 11. The lead-out port 12 may be used as a lumen for a guide wire for guiding the catheter device 1F into the living body. Further, the guide portion 40 can be disposed at an appropriate position based on the arrangement position of the lead-out port 12. For example, as illustrated in FIG. 13, when the guide portion 40 is disposed below the first lead-out port 121, the urine 310 flowing in from the first lead-out port 121 can flow toward the sensor unit 60 without stagnation after coming into contact with the guide portion 40.

Since the catheter device 1F of the sixth modification includes the plurality of lead-out ports 12, which are the first lead-out port 121 and the second lead-out port 122, it is possible to efficiently allow the urine 310 to flow into the lead-out lumen 11. In addition, since the catheter device 1F includes the guide portion 40, the urine 310 is guided toward the sensor unit 60 without stagnation from the lead-out port 12 to the sensor unit 60, and the urine 310 can be constantly brought into contact with the sensor unit 60.

As described above, the catheter device 1 according to the present embodiment has an elongated main body portion 10 having a lead-out lumen 11 that is indwelled in a living body and through which a biological fluid (urine 310) can flow. The main body portion 10 includes a lead-out port 12 that allows the inside and the outside of the lead-out lumen 11 to communicate, one or more sensor units 60 capable of measuring a component contained in the urine 310 in the lead-out lumen 11, and a guide portion 40 that guides the urine 310 flowing into the lead-out lumen 11 from the lead-out port 12 toward the sensor unit 60 side. The guide portion 40 protrudes in the radial direction orthogonal to the central axis C of the main body portion 10, and is disposed on the distal end side relative to the sensor unit 60, preferably on the proximal end side relative to the distal end side end portion of the lead-out port 12, and on the distal end side relative to the sensor unit 60.

With such a configuration, the urine 310 flowing in from the lead-out port 12 is guided to the sensor unit 60 side without stagnation after coming into contact with the guide portion 40, and constantly comes into contact with the sensor surface. Therefore, the catheter device 1 can stably measure the urine 310 immediately after flowing in from the lead-out port 12.

The detailed description above describes embodiments of a catheter device representing examples of the new catheter device disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents that fall within the scope of the claims are embraced by the claims.

### REFERENCE SIGNS LIST

11A to 1F Catheter device
10 Main body portion
11 Lead-out lumen
12 Lead-out port
13 Sensor lumen (13a blocking portion)
14 Fluid lumen
20 Indwelling portion
21 Expansion/contraction lumen
30 Distal end tip
40, 40A to 40E Guide portion
50 Hub
51 First branch portion
52 Second branch portion
60 Sensor unit
61 Oxygen sensor (61a oxygen sensor main body, 61b oxygen transmission unit,
61c measurement unit, 61d substrate, 61e phosphor)
62 Temperature sensor (62a temperature sensor main body, 62b temperature transmission unit)
70 Fixing portion
200 Measurement device
300 Bladder
310 Urine
400 Urine collection bag
410 Urine collection tube
C Central axis

What is claimed is:

1. An elongated catheter device extending in an axial direction and configured to be indwelled in a living body, comprising:
a lead-out lumen through which a biological fluid is flowable while the elongated catheter device is indwelled in the living body, the lead-out lumen extending in the axial direction;
a lead-out port that communicates the lead-out lumen with an outside of the lead-out lumen so that the biological fluid in the living body flows from the outside of the lead-out lumen into the lead-out lumen by way of the lead-out port while the elongated catheter device is indwelled in the living body;
one or more sensor units each configured to measure a component contained in the biological fluid flowing in the lead-out lumen while the elongated catheter device is indwelled in the living body;
a guide portion that guides the biological fluid flowing into the lead-out lumen from the lead-out port toward a side of the sensor unit; and
the guide portion protruding in a radial direction orthogonal to a central axis of the catheter device in a longitudinal direction, and disposed on a distal end side relative to the sensor unit.

2. The catheter device according to claim 1, wherein the guide portion is disposed on a proximal end side relative to a distal end side end portion of the lead-out port and on a distal end side relative to the sensor unit.

3. The catheter device according to claim 1, wherein the catheter device includes a central axis extending along a length of the catheter device, the guide portion being disposed on a same side of the catheter device as the lead-out port as seen in a longitudinal cross-sectional view taken along a plane that contains the central axis of the catheter device.

4. The catheter device according to claim 1, wherein the catheter device includes a central axis extending along a length of the catheter device, the guide portion being disposed on a side of the catheter device opposite to the lead-out port as seen in a longitudinal cross-sectional view taken along a plane that contains the central axis of the catheter device.

5. The catheter device according to claim 1, wherein the guide portion is disposed at a position that is not overlapping with a distal end of the sensor unit in the axial direction.

6. The catheter device according to claim 1, wherein the guide portion has a length in which the guide portion intersects the central axis of the catheter device in a longitudinal cross-sectional view taken along a plane that contains the central axis of the catheter device.

7. The catheter device according to claim 1, wherein
the guide portion includes an upper surface facing a distal end side and a lower surface facing a proximal end side, and
at least a central axis side end portion of the upper surface is inclined toward the proximal end side.

8. The catheter device according to claim 7, wherein the upper surface of the guide portion has a guide structure that guides the biological fluid flowing in from the lead-out port to the sensor unit side.

9. The catheter device according to claim 1, wherein
the guide portion includes a wall covering the lead-out lumen and a through hole in the wall that allows communication between the lead-out lumen on a distal end side of the guide portion and the lead-out lumen on a proximal end side of the guide portion, and
the through hole being disposed immediately above the sensor unit.

10. The catheter device according to claim 1, further comprising
a sensor lumen that liquid-tightly accommodates the sensor unit;
the sensor unit including a first sensor unit partially exposed from the sensor lumen so as to be contactable with the biological fluid, and a second sensor unit liquid-tightly accommodated in the sensor lumen.

11. The catheter device according to claim 10, wherein
the sensor lumen includes a first sensor lumen that accommodates the first sensor unit with at least a part of the first sensor unit exposed, and a second sensor lumen that liquid-tightly accommodates the second sensor unit, and
the guide portion is either a first blocking portion that blocks a distal end opening of the first sensor lumen or a second blocking portion that blocks a distal end opening of the second sensor lumen.

12. The catheter device according to claim 1, further comprising a fixing portion that fixes the sensor unit, wherein the fixing portion functions as the guide portion.

13. The catheter device according to claim 1, wherein the catheter device includes a central axis extending along a length of the catheter device, wherein the guide portion includes a first guide portion and a second guide portion, the second guide portion being disposed on a distal end side relative to the first guide portion and on a side of the catheter device opposite to the first guide portion with respect to the central axis as seen in a longitudinal cross-sectional view taken along a plane that contains the central axis of the catheter device.

14. The catheter device according to claim 1, wherein the catheter device includes a central axis extending along a length of the catheter device, wherein the lead-out port includes a first lead-out port disposed on a same side of the central axis as the guide portion and a second lead-out port disposed on a side of the central axis opposite to the guide portion as seen in a longitudinal cross-sectional view taken along a plane that contains the central axis of the catheter device.

15. The catheter device according to claim 1, wherein the sensor unit includes a fluorescent sensor configured to measure at least one of: an oxygen partial pressure in the biological fluid, a carbon dioxide partial pressure in the biological fluid, an ion concentration in the biological fluid, a sodium ion in the biological fluid, a potassium ion in the biological fluid, glucose in the biological fluid, lactic acid in the biological fluid, and an antibody-added protein in the biological fluid.

16. The catheter device according to claim 1, further comprising
an indwelling portion that is indwelled in the living body and is configured to expand and contract,
the guide portion being disposed on a distal end side relative to a proximal end side end portion of the indwelling portion.

17. The catheter device according to claim 1, further comprising
a sensor lumen that liquid-tightly accommodates the sensor unit,
the sensor unit being insertable into and removable from the sensor lumen.

18. The catheter device according to claim 1, further comprising
a sensor lumen that liquid-tightly accommodates the sensor unit,
the sensor lumen accommodating a transmission unit that is connected to and extends away from the sensor unit, and
the transmission unit including at least one or more transmission unit connection points.

19. A catheter device configured to be indwelled in a living body, the catheter device comprising:
an elongated tubular body having a distal end, the elongated tubular body including a lead-out lumen through which biological fluid from the living is flowable while the catheter device is indwelled in the living body, the lead-out lumen extending along a longitudinal extent of the elongated tubular body;
a lead-out port passing through the wall of the elongated tubular body and communicating the lead-out lumen with an outside of the elongated tubular body so that the biological fluid in the living body flows from the outside of the elongated tubular body into the lead-out lumen by way of the lead-out port while the catheter device is indwelled in the living body;
a sensor configured to measure a component contained in the biological fluid flowing in the lead-out lumen while the catheter device is indwelled in the living body, the sensor being positioned in the lead-out lumen at a position on a proximal side of the distal end of the elongated tubular body;
a transmission unit connected to the sensor to convey information from the sensor to outside the catheter device; and
a guide portion: i) positioned in the lead-out lumen; ii) located on the proximal side of the lead-out port at a position to be contacted by the biological fluid flowing through the lead-out port and entering the lead-out lumen; iii) located on a distal side of the sensor; iv) protruding radially inwardly away from an inner surface of the elongated tubular body; and v) configured to guide the biological fluid flowing through the lead-out port, entering the lead-out lumen and contacting the guide portion toward the sensor unit.

20. A method comprising:
inserting a catheter device into a biological lumen of a living body containing a biological fluid, the catheter device comprising: a lead-out port; an axially extending lead-out lumen in fluid communication with the lead-out port; a sensor configured to measure a component contained in the biological fluid; and a guide portion;
the biological fluid in the biological lumen passing through the lead out port and being introduced into the lead out lumen after passing through the lead out port; and
the biological fluid that is introduced into the lead out lumen by way of the lead out port contacting the guide portion that projects radially inwardly toward a central axis of the elongated catheter device and being guided by the guide portion toward the sensor.
